(19) United States
(12) Patent Application Publication
(10) Pub. No.: US 2012/0022610 A1
(43) Pub. Date: Jan. 26, 2012

(54) Devices for Delivering Neuro Electro Adaptive Therapy NEAT

(76) Inventors: Kenneth Blum, San Diego, CA (US); Roger L. Waite, San Diego, CA (US); Bernard William Downs, Lederach, PA (US); William J. Heaney, Huntington Beach, CA (US)

(21) Appl. No.: 13/092,909

(22) Filed: Apr. 22, 2011

Related U.S. Application Data
(60) Provisional application No. 61/326,755, filed on Apr. 22, 2010.

(51) Int. Cl. A61N 1/372

(52) U.S. Cl. 607/45

(57) ABSTRACT

A new class of therapeutic neuro-electro-adaptive devices for delivering cranial electrotherapy (NEAT) to patients for the treatment of various diseases and disorders, including those involving Reward Deficiency Syndrome (RDS), are described, as are various methods for using such devices, for example, to treat RDS behaviors.

### RELATED APPLICATION

This application claims the benefit of and priority to U.S. provisional patent application Ser. No. 61/326,755, filed 22 Apr. 2010, the contents of which are hereby incorporated in their entirety for any and all purposes.

### BACKGROUND OF THE INVENTION

1. Field of the Invention

The present invention relates generally to neuro-electro-adaptive devices for delivering cranial electrotherapy (NEAT) to patients.

2. Overview.

The following description includes information that may be useful in understanding the present invention. It is not an admission that any such information is prior art, or relevant, to the presently claimed inventions, or that any publication specifically or implicitly referenced is prior art.

The Cranial Electrical Stimulation (CES) technique appeared at the beginning of the 1960s and is aimed to act at the level of the central nervous system. The current, composed of high frequency pulses interrupted with a repetitive low frequency, was delivered through three electrodes (a negative electrode placed between the eyebrows while two positive electrodes are located in the retro-mastoid region on each side of a patient's head). Shortcomings encountered with previous electrical stimulation techniques were avoided due to changes in the characteristics of the delivered current. The main property of CES is to potentiate some drug effects, especially opiates and neuroleptics, during anesthetic clinical procedures. Such potentiation permits a drastic reduction of pharmacological anesthetic and results in reduced post-operative complications. Despite numerous clinical and animal studies performed with this technique for several decades, CES mechanisms are not completely elucidated. Animal studies demonstrated that stimulation with CES releases 5-hydroxy-indol-acetic acid and enkephalins without any undesirable outcome. Despite these encouraging results, the use of CES in Substance Use Disorder (SUD) or Reward Deficiency Syndrome (RDS) has been fraught with mixed results.

Brief History

CES received U.S. Food and Drug Administration (FDA) approval for the treatment of insomnia, depression, and anxiety in 1979. CES is the U.S. FDA term for the transcranial application of small amounts of electricity, usually less than 300-600 mA with a frequency of 100 Hz or lower. CES was imported into the United States from Europe and was originally introduced as electro sleep, possibly because it increases delta waves. CES and electronic medicine did not receive a particularly warm reception in American medicine until clinicians began to utilize the transcutaneous electrical nerve stimulation (TENS) devices for pain. Electrotherapies have been used in psychiatry in the form of electroconvulsive therapy (ECT), which is still utilized in organic brain diseases such as Parkinson's and organic-based depressions.

As such, there is a continuing need to provide additional and more effective treatments for SUD and RDS. This invention address that need.

3. Definitions.

When used in this specification, the following terms will be defined as provided below unless otherwise stated. All other terminology used herein will be defined with respect to its usage in the particular art to which it pertains unless otherwise noted.

A “patentable” composition, process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that (1) preserves their validity and (2) provides the broadest reasonable interpretation under the circumstances.

A “plurality” means more than one.

### BRIEF DESCRIPTION OF THE DRAWING

A brief summary of the drawing is provided below.

FIG. 1 illustrates the NEAT-12 device.

### DESCRIPTION OF THE INVENTION

The object of the invention is to provide new, patentable neuro-electro-adaptive devices for delivering cranial electrotherapy (NEAT) to patients for use in the treatment of insomnia, depression, anxiety, and addiction as described in Reward Deficiency Syndrome (RDS). The NEAT devices of the invention are portable, battery powered cranial electrotherapy stimulators. The devices apply an electrical current to a patient's head, through electrodes clipped to the patient's ear lobes. Electrical leads connect the electrodes to the pulse generator, which is in turn controlled by a processor executing software or computer control logic configured to result in the delivery from the device of the desired electrostimulation regimen.

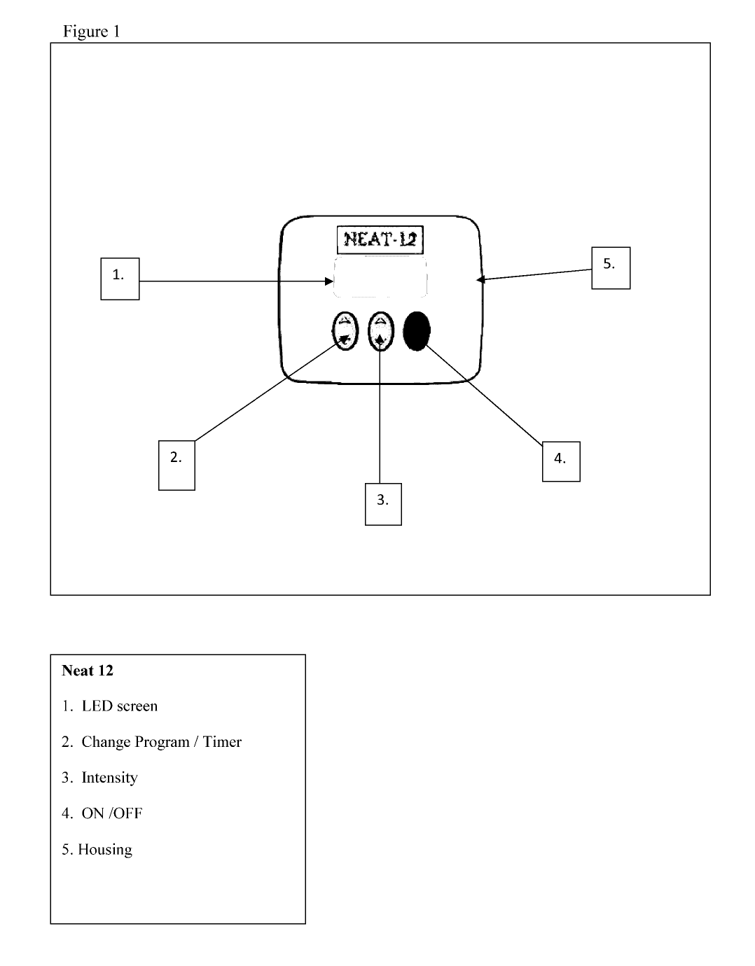

A preferred embodiment, termed the NEAT-12 device, is shown in FIG. 1. The NEAT-12 device has a housing (5) constructed of lightweight ABC plastic. The device has an LED screen (1) and three control buttons, one to change programs and/or timing (2), one to adjust stimulation (i.e., signal) intensity (3), and an “On/Off” switch (4). Here, intensity generally refers to voltage, but can also refer to current, depending on context. The dimensions of the device are approximately 2.875″ L×2.125″ W×0.75″ H and weighs approximately 3 ozs. There is one lead wire (not shown), which is approximately 40″ long and weighs approximately 3 ozs. The lead wire and electrodes (not shown) comply with the performance standard set forth in 21 CFR 898. Preferably, the electrodes are self-adhesive or other easily attachable to the ear lobe of a patient. The display (1) shows information such as program number, current frequency, voltage settings, time, battery condition, etc.

The therapeutic electrostimultion devices of the invention have memories that store a plurality of unique programmed therapeutic waveforms for use in a treatment session. The waveforms are comprised of alternating positive voltage pulses and negative voltage pulses, wherein the frequency of the pulses and of the bipolar modulation are both fixed. The pulse widths of the waveforms are either fixed or variable, depending on the program selected. The pulses are comprised of square waves coupled to the output terminals through a parallel resistor/capacitor combination that results in a bi-phasic exponential decaying waveform. In the practice of this invention waveforms of this variety typically have a frequency between about 0.01 Hz and about 10 Hz, a voltage between about 0.01 volt (V) and 75 volts, a current between about 0.01 microampere and about 125 microampere, and a pulse duration of between about 10 milliseconds and 5 seconds, as such waveforms have unexpectedly been discovered to have beneficial effects on RDS behaviors, among others.

In one preferred embodiment of this aspect, the device is programmed to deliver a bi-phasic square waveform in resting mode without a resistive load, which changes to an exponential decaying waveform with the same waveform characteristics under resistance. Preferably, the frequency of such waveform is 0.01-10 Hz with a polarity change of 0.01-3 s (each pulse) and a related pulse width of 0.01-5 s (50% duty).

In one preferred embodiment, the device is programmed to deliver a square waveform with bi-phasic modulation in resting mode without a resistive load, which changes to an exponential decaying waveform with same waveform characteristics under resistance. Preferably, the frequency of such waveform is 0.01-10 Hz with a polarity change of 0.01-3 s (every 6 pulses) and a related pulse width of 0.01-3 s (50% duty).

In one preferred embodiment, the device is programmed to deliver a square waveform with bi-phasic modulation in resting mode without a resistive load, which and changes to an exponential decaying waveform with same waveform characteristics under resistance. Preferably, the frequency of such waveform is 0.01-10 Hz with a polarity change of 0.01-3 s (every 17 pulses) and a related pulse width of 0.01-3 s (50% duty).

In one preferred embodiment, the device is programmed to deliver a square waveform with randomized pulse width modulation in resting mode without a resistive load, which changes to an exponential decaying waveform with same waveform characteristics under resistance. Preferably, the frequency of such waveform is 0.01-10 Hz with a polarity change of 0.01-3 s (every pulse) and a randomized modulated pulse width varying dependant to resistance.

In one preferred embodiment, the device is programmed to deliver a square waveform with randomized pulse width modulation and bi-phasic modulation in resting mode without a resistive load, which changes to an exponential decaying waveform with same waveform characteristics under resistance. Preferably, the frequency of such waveform is 0.01-10 Hz with a polarity change of 0.01-3 s (every 6 pulses) and a randomized modulated pulse width varying dependant to resistance.

In one preferred embodiment, the device is programmed to deliver a square waveform with randomized pulse width modulation and bi-phasic modulation in resting mode without a resistive load, which changes to an exponential decaying waveform with same waveform characteristics under resistance. Preferably, the frequency of such waveform is 0.01-10 Hz with a polarity change of 0.01-3 s (every 17 pulses) and a randomized modulated pulse width varying dependant to resistance.

In various preferred embodiments, the devices deliver 0.01-125 μA with a maximum output of 0.01-75V pk-pk @ 500 Ohms, 2 Kohms, and 10 KOhms. Randomized pulse widths range between 0.01 ms to 1.90 s depending upon setting.

In one preferred embodiment, the device is programmed to deliver a bi-phasic qquare waveform in resting mode without a resistive load, which changes to an exponential decaying waveform with same waveform characteristics under resistance. Preferably, the frequency of such waveform is 0.01-10 Hz with a polarity change of 0.01-3 s (every pulse) and a related pulse width of 0.01-3 s (50% duty).

In one preferred embodiment, the device is programmed to deliver a bi-phasic qquare waveform in resting mode without a resistive load, which changes to an exponential decaying waveform with same waveform characteristics under resistance. Preferably, the frequency of such waveform is 0.01-10 Hz with a polarity change of 0.01-3 s (every 6 pulses) and a related pulse width of 0.01-3 s (50% duty).

In one preferred embodiment, the device is programmed to deliver a square waveform with bi-phasic modulation in resting mode without a resistive load, which changes to an exponential decaying waveform with same waveform characteristics under resistance. Preferably, the frequency of such waveform is 0.01-10 Hz with a polarity change of 0.01-3 s (every 17 pulses) and a related pulse width of 0.01-3 s (50% duty).

In one preferred embodiment, the device is programmed to deliver a bi-phasic square waveform with randomized pulse width modulation in resting mode without a resistive load, which changes to an exponential decaying waveform with same waveform characteristics under resistance. Preferably, the frequency of such waveform is 0.01-10 Hz with a polarity change of 0.01-3 s (every pulse) and a randomized modulated pulse width varying dependant to resistance.

In one preferred embodiment, the device is programmed to deliver a square waveform with randomized pulse width modulation and bi-phasic modulation in resting mode without a resistive load, which changes to an exponential decaying waveform with same waveform characteristics under resistance. Preferably, the frequency of such waveform is 0.01-10 Hz with a polarity change of 0.01-3 s (every 6 pulses) and a randomized modulated pulse width varying dependant to resistance.

In one preferred embodiment, the device is programmed to deliver a square waveform with randomized pulse width modulation and bi-phasic modulation in resting mode without a resistive load, which changes to an exponential decaying waveform with same waveform characteristics under resistance. Preferably, the frequency of such waveform is 0.01-10 Hz with a polarity change of 0.01-3 s (every 17 pulses) and a randomized modulated pulse width varying dependant to resistance.

In various preferred embodiments, certain of the devices deliver 0.1-112 μA with a maximum output of 0.01-75V pk-pk (peak-to-peak) @ 500 Ohms, 2 Kohms, and 10 KOhms. Randomized pulse widths range between 0.01 ms to 1.90 s depending upon setting.

In one preferred embodiment, the device is programmed to deliver a bi-phasic square waveform in resting mode without a resistive load, which changes to an exponential decaying waveform with same waveform characteristics under resistance. Preferably, the frequency of such waveform is 0.44 Hz with a polarity change of 2.25 s (each pulse) and a related pulse width of 1.25 s (50% duty).

In one preferred embodiment, the device is programmed to deliver a bi-phasic square waveform in resting mode without a resistive load, which changes to an exponential decaying waveform with same waveform characteristics under resistance.

In one preferred embodiment, the device is programmed to deliver a square waveform with bi-phasic modulation in resting mode without a resistive load, which changes to an exponential decaying waveform with same waveform characteristics under resistance. Preferably, the frequency of such waveform is 2.5 Hz with a polarity change of 2.4 s (every 6 pulses) and a related pulse width of 0.2 s (50% duty).

In one preferred embodiment, the device is programmed to deliver a square waveform with bi-phasic modulation in resting mode without a resistive load, which changes to an exponential decaying waveform with same waveform characteristics under resistance. Preferably, the frequency of such waveform is 7 Hz with a polarity change of 2.428 s (every 17 pulses) and a related pulse width of 0.0714 s (50% duty).

In one preferred embodiment, the device is programmed to deliver a bi-phasic square waveform with randomized pulse width modulation in resting mode without a resistive load, which changes to an exponential decaying waveform with same waveform characteristics under resistance. Preferably, the frequency of such waveform is 0.44 Hz with a polarity change of 2.25 s (every pulse) and a randomized modulated pulse width varying dependant to resistance.

In one preferred embodiment, the device is programmed to deliver a square waveform with randomized pulse width modulation and bi-phasic modulation in resting mode without a resistive load, which changes to an exponential decaying waveform with same waveform characteristics under resistance. Preferably, the frequency of such waveform is 2.5 Hz with a polarity change of 2.4 s (every 6 pulses) and a randomized modulated pulse width varying dependant to resistance.

In one preferred embodiment, the device is programmed to deliver a square waveform with randomized pulse width modulation and bi-phasic modulation in resting mode without a resistive load, which changes to an exponential decaying waveform with same waveform characteristics under resistance. Preferably, the frequency of such waveform is 7 Hz with a polarity change of 2.428 s (every 17 pulses) and a randomized modulated pulse width varying dependant to resistance.

In various embodiments, certain of the devices deliver 0.1-125 μA with a maximum output of 0.01-75V pk-pk (peak-to-peak) @ 500 Ohms, 2 Kohms, and 10 KOhms. Randomized pulse widths range between 0.01 ms to 1.90 s depending upon setting.

In one preferred embodiment, the device is programmed to deliver a bi-phasic square waveform in resting mode without a resistive load, which changes to an exponential decaying waveform with same waveform characteristics under resistance. Preferably, the frequency of such waveform is 0.44 Hz with a polarity change of 2.25 s (each pulse) and a related pulse width of 1.25 s (50% duty).

In one preferred embodiment, the device is programmed to deliver a square waveform with bi-phasic modulation in resting mode without a resistive load and changes to an exponential decaying waveform with same waveform characteristics under resistance. Preferably, the frequency of such waveform is 2.5 Hz with a polarity change of 2.4 s (every 6 pulses) and a related pulse width of 0.2 s (50% duty).

In one preferred embodiment, the device is programmed to deliver a square waveform with bi-phasic modulation in resting mode without a resistive load and changes to an exponential decaying waveform with same waveform characteristics under resistance. Preferably, the frequency of such waveform is 7 Hz with a polarity change of 2.428 s (every 17 pulses) and a related pulse width of 0.0714 s (50% duty).

In one preferred embodiment, the device is programmed to deliver a bi-phasic square waveform with randomized pulse width modulation in resting mode without a resistive load, which changes to an exponential decaying waveform with same waveform characteristics under resistance. Preferably, the frequency of such waveform is 0.44 Hz with a polarity change of 2.25 s (every pulse) and a randomized modulated pulse width varying dependant to resistance.

In one preferred embodiment, the device is programmed to deliver a square waveform with randomized pulse width modulation and bi-phasic modulation in resting mode without a resistive load, which changes to an exponential decaying waveform with same waveform characteristics under resistance. Preferably, the frequency of such waveform is 2.5 Hz with a polarity change of 2.4 s (every 6 pulses) and a randomized modulated pulse width varying dependant to resistance.

In one preferred embodiment, the device is programmed to deliver a square waveform with randomized pulse width modulation and bi-phasic modulation in resting mode without a resistive load, which changes to an exponential decaying waveform with same waveform characteristics under resistance. Preferably, the frequency of such waveform is 7 Hz with a polarity change of 2.428 s (every 17 pulses) and a randomized modulated pulse widthvarying dependant to resistance.

In various embodiments, certain of the devices deliver 100 μA with a maximum output of 48V pk-pk @ 500 Ohms, 2 Kohms, and 10 KOhms. Randomized pulse widths range between 24 ms to 2.5 s depending upon setting.

In one preferred embodiment, the device is programmed to deliver a non-modulated signal wherein the pulse width or duration of individual pulses ranges from about 1 microsecond to 1000 microseconds measured with a 1000 ohm load.

Another aspect relates to methods of using the devices of the invention, for example, to treat RDS behaviors and/or behavioral symptoms, addictive disorders, impulsivity, compulsivity, and obsessive-compulsive behaviors. RDS behaviors that can be treated with these devices include alcoholism, psychostimulant addication, opiate (heroin and prescription drugs) addiction, nicotine (smoking behavior) addiction, benzodiazepine use, depressant (barbiturates) abue, cannabis use, bath salt, spice, glucose, food, and carbohydrate bingeing, SUD, spectrum disorders, ADHD, Tourette's syndrome, autism, pathological gambling, sexual addiction, immature defense styling (lying), gaming addiction (e.g. video), psychedelic drug use, psychoactive mushroom use, excessive shopping, stealing, domestic violence, aggression, murder, and depression (e.g. bipolar, major depression, schizophrenia, anxiety, personality disorders, dysthymia, and borderline).

Without wishing to be bound to any particular theory, the inventors believe that the mechanism by which the devices of the invention provide a therapeutic benefit is through device-mediated stimulation of the pre-frontal cortex and particularly the cingulate gyrus of a patient's brain. This is believed to occur because in addicted individuals there is a hypodopaminergic state in the reward system. In the mesolimbic region it is know that carriers of the DRD2 gene A1 allele have a 30-40% reduction in D2 receptors, which alone is known to increase abnormal craving behavior. Relapse is also tied to hypodopaminergic function in the prefrontal cortex. The main target area responsible for relapse is the pre-frontal cortex and Cingulate Gyrus. It appears that the devices of the invention uniquely cause normalization of the widespread Theta waves in the relapse target area (Cingulate Gyrus), especially during protracted abstinence. It is known from the literature that using biofeedback to treat alcoholics requires about 20 sessions, which results in an increase in alpha waves and increase in low beta bands (calming). Similar results can be achieved one hour after treatment with a device according to the invention, such as the NEAT-12. Accordingly, the devices of the invention can also be beneficially used to prevent breakthrough cravings.

### DESCRIPTION OF A PREFERRED EMBODIMENT

The following description concerns a particularly preferred embodiment of a device according to the invention, referred to herein as the NEAT-12 device. See FIG. 1. The electric parameters for the NEAT-12 device are shown in Table 1, below.

TABLE 1
NEAT-12 Parameters

| Parameter | Value | Calculation |
|---|---|---|
| Maximum voltage: | 48 Vₚₖ₋ₚₖ | By design |
| | (±24 V) | |
| Maximum current: | 96 mAₚₖ₋ₚₖ | 24V ÷ 500 ohm = 48 mA |
| | (±48 mA) | |
| Minimum frequency: | 0.44 Hz | By design |
| Maximum frequency: | 7 Hz | By design |
| Minimum pulse width: | 24 ms | (1/7 Hz) × 16.7% |
| | | modulation |
| Maximum pulse width: | 1.893 s | (1/0.44 Hz) × 83.3% |
| | | modulation |
| Phase charge: | 2.4 μcoulombs | 0.1 μF × 24 V |
| Phase energy: | 29 μW | 0.1 μF × (24 V)² ÷ 2 |
| Clip contact area: | 0.866 cm² | (1.05 cm)² × π/4 |
| Maximum current | 39 μA/cm² | 7 Hz × 2 × 2.4 μC ÷ 0.866 cm² |
| density: | | |
| Maximum power | 469 μW/cm² | 7 Hz × 2 × 29 μW ÷ 0.866 cm² |
| density: | | |

The memory of the NEAT-12 device stores data for 12 different therapeutic waveforms and pulse sequences, each of which is described in Table 2, below.

TABLE 2
NEAT-12 programs

| | | | | *Pulse | |
|---|---|---|---|---|---|
| Program | Name | MicroAmperage | Frequency | Width | Comment |
| 1 | Low Freq | 25 | 0.44 Hz | 1.12 s | This program is for those individuals that have a simple diagnosis |
| | Steady | | | | of stress, anxiety or insomnia in aftercare treatment. |
| 2 | Med Freq | 25 | 2.5 Hz | .02 s | This program is for those individuals that have a moderate |
| | Steady | | | | diagnosis of stress, anxiety or insomnia in aftercare treatment. |
| 3 | High Freq | 25 | 7 Hz | 0.714 s | This program is for those individuals that have a severe |
| | Steady | | | | diagnosis of stress, anxiety or insomnia in aftercare treatment. |
| 4 | Low Freq | 25 | 0.44 Hz | High 1.85 s | This program is for those individuals that have a simple diagnosis |
| | modulated | | | Low .33 s | of stress, anxiety or drug addition in aftercare treatment. |
| 5 | Med Freq | 25 | 2.5 Hz | High .32 s | This program is for those individuals that have a moderate |
| | modulated | | | Low .06 s | diagnosis of stress, anxiety or drug addition in aftercare |
| | | | | | treatment. |
| 6 | High Freq | 25 | 7 Hz | High .114 s | This program is for those individuals that have a severe |
| | modulated | | | Low .021 s | diagnosis of stress, anxiety or drug addition in aftercare |
| | | | | | treatment. |
| 7 | Low Freq | 100 | 0.44 Hz | 1.12 s | This program is for those individuals that have a simple diagnosis |
| | Steady | | | | of stress, anxiety or insomnia that are in acute treatment. |
| 8 | Med Freq | 100 | 2.5 Hz | .02 s | This program is for those individuals that have a moderate |
| | Steady | | | | diagnosis of stress, anxiety or insomnia that are in acute |
| | | | | | treatment. |
| 9 | High Freq | 100 | 7 Hz | 0.714 s | This program is for those individuals that have a severe diagnosis |
| | Steady | | | | of stress, anxiety or insomnia that are in acute treatment. |
| 10 | Low Freq | 100 | 0.44 Hz | High 1.85 s | This program is for those individuals that have a simple diagnosis |
| | modulated | | | Low .33 s | of stress, anxiety or drug addition that are in acute treatment. |
| 11 | Med Freq | 100 | 2.5 Hz | High .32 s | This program is for those individuals that have a moderate |
| | modulated | | | Low .06 s | diagnosis of stress, anxiety or drug addition that are in acute |
| | | | | | treatment. |
| 12 | High Freq | 100 | 7 Hz | High .114 s | This program is for those individuals that have a severe diagnosis |
| | modulated | | | Low .021 s | of stress, anxiety or drug addition that are in acute treatment. |

*The measurement of the pulse width reported is without a load. Also, in a non-modulated program the pulse width measurement of individual pulses, the pulse duration range is 1 microsecond to 1000 microseconds with a 1000 ohm load.

Although the invention has been described with reference to the above specification, it will be understood that modifications and variations are encompassed within the spirit and scope of the invention. Accordingly, the invention is limited only by the appended claims.

All of the articles, devices, compositions, kits, and methods described and claimed herein can be made and executed without undue experimentation in light of the present specification. While the articles, devices, compositions, kits, and methods of this invention have been described in terms of preferred embodiments, it will be apparent to those of skill in the art that variations may be applied to the articles, devices, compositions, kits, and methods and in the steps or in the sequence of steps of the methods described herein without departing from the spirit and scope of the invention as defined by the appended claims.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A therapeutic electrostimultion device configured to deliver a plurality of waveforms to a patient, comprising in operable association:
a. a pulse generator capable of sequentially delivering each of a plurality of therapeutic waveforms;
b. a memory storing data representing a plurality of therapeutic waveforms and pulse sequences, wherein the plurality of therapeutic waveforms includes a first waveform that comprises a fixed width and a second waveform that comprises an exponentially decaying waveform;
c. a processor configured to control delivery by the pulse generator of at least two of plurality of pulse sequences to a patient, wherein at least one of the plurality of pulse sequences comprises the second waveform;
d. a pair of electrodes adapted for attachment to the ears of a patient; and
e. a power supply.

2. A device according to claim 1 wherein a bi-phasic qquare waveform in resting mode without a resistive load changes to an exponential decaying waveform with same waveform characteristics under resistance.

3. A device according to claim 1 wherein a square waveform with bi-phasic modulation in resting mode without a resistive load changes to an exponential decaying waveform with same waveform characteristics under resistance.
